(19) United States
(12) Patent Application Publication
(10) Pub. No.: US 2025/0041371 A1
(43) Pub. Date: Feb. 6, 2025

(54) Compositions And Methods For Fat Reduction

(71) Applicant: Bellasyn, LLC, Palm Harbor, FL (US)

(72) Inventors: Diane I. Duncan, Fort Collins, CO (US); Steve M. Kushner, Oldsmar, FL (US)

(73) Assignee: Bellasyn, LLC, Palm Harbor, FL (US)

(21) Appl. No.: 18/782,637

(22) Filed: Jul. 24, 2024

Related U.S. Application Data
(60) Provisional application No. 63/529,996, filed on Jul. 31, 2023.

(51) Int. Cl. A61K 36/424; A61K 9/00; A61K 36/258; A61K 36/481; A61K 36/537; A61K 36/605; A61K 36/815; A61K 36/9064; A61K 47/10; A61K 47/26; A61K 47/32; A61K 47/44; A61P 3/04

(52) U.S. Cl. CPC A61K 36/424; A61K 9/0014; A61K 36/258; A61K 36/481; A61K 36/537; A61K 36/605; A61K 36/742; A61K 36/815; A61K 36/9064; A61K 47/10; A61K 47/26; A61K 47/32; A61K 47/44; A61P 3/04

(57) ABSTRACT

Generally, compositions effective for the reduction of fat deposition and/or enhancement of muscle mass. In particular, compositions including admixtures of dry ingredients or admixtures of dry ingredients combined with a carrier composition to aid delivery of constituents of the admixture of dry ingredients.

This United States Non-Provisional patent application claims the benefit of U.S. Provisional Patent Application No. 63/529,996, filed Jul. 31, 2023, hereby incorporated by reference herein.

### I. FIELD OF THE INVENTION

Generally, compositions that are effective for the reduction of fat deposition and/or to preserve or enhance muscle mass. In particular, compositions including admixtures of dry ingredients or admixtures of dry ingredients combined with a carrier composition to aid delivery of constituents of the admixture of dry ingredients.

### II. BACKGROUND

Sarcopenic obesity is characterized by the coexistence of obesity, characterized as >35 percent body fat, and sarcopenia, determined by low skeletal muscle mass and function. Donini L. et al. Definition and diagnostic criteria for sarcopenic obesity: ESPEN and EASO Consensus Statement. Obes Facts. 15 (3) (2022), pp. 321-335. <doi.org/10.1159/000521241>.

However, the condition can be difficult to attribute an exact definition due to the varying norms of different cultures. In the United States, the prevalence of sarcopenic obesity is reported at about 18.1% in women over 60 and about 42.9% in similarly aged men. Shafiee G., Keshtkar A., Soltani A., Ahadi Z., Larijani B., Heshmat R. Prevalence of sarcopenia in the world: a systematic review and meta-analysis of general population studies. J. Diabetes Metab Disord. 16 (2017), pp. 16:21. <doi: 10.1186/s40200-017-0302-x>.

Basal metabolic rate can decline by over 4% per decade after age 50. Zoico E., Di Francesco V., Mazzali G., Vettor R., Fantin F. et al. Adipocytokines, fat distribution, and insulin resistance in elderly men and women. J Gerontol A Biol Sci Med Sci 59 (2004), pp. 935-939.

The rate of skeletal muscle loss can vary between 3% to 8% every ten years after skeletal muscle mass peaks in early adulthood. Sarcopenic obesity is frequently observed among the elderly population, with both its risk and prevalence rising as individuals age. Johnson Stoklossa C. A. et al. Prevalence of Sarcopenic Obesity in Adults with Class II/III Obesity Using Different Diagnostic Criteria [online]. J Nutr Metab (2017) [retrieved on Jul. 18, 2023]. Retrieved from Internet: <https://www.ncbi.nlm.nih.gov/pmc/articles/PMC5380855/><doi: 10.1155/2017/7307618>.

In subjects older than 65, the rate of loss can accelerate with a range of 6% to 15% prevalence of clinical sarcopenia. Vincent H. K., Raiser S. N., Vincent K. R. The aging musculoskeletal system and obesity-related considerations with exercise. Ageing Res Rev. 11 (3) (2012 Mar. 15), pp. 361-373. <doi: 10.1016/j.arr.2012.03.002>.

The general deterioration of the musculoskeletal system leads to negative consequences, including increased likelihood of falls, reduced functionality, increased frailty, and mortality. These outcomes can be associated with a decrease in quality of life and an elevated risk of cardiometabolic conditions such as diabetes and hypertension. Baumgartner R. N., Wayne S. J., Waters D. L., Janssen I., Gallagher D., Morley J. E. Sarcopenic obesity predicts instrumental activities of daily living disability in the elderly. Obes. Res. 12 (2004), pp. 1995-2004.

Visceral fat area greater than 100 cm² in both women and men characterizes visceral obesity. Lim S., Kim J. H. et al. Sarcopenic obesity: prevalence and association with metabolic syndrome in the Korean Longitudinal Study on Health and Aging (KLOSHA). Diabetes Care 33 (2010), pp. 1652-1654.

A centralization of fat from the subcutaneous to visceral deposition peaks between the ages of 60 and 75. Excess calories previously converted to subcutaneous fat are then stored in the intra-abdominal space. Proinflammatory cytokines and free fatty acids released from VAT cause dyslipidemia and insulin resistance. Stenholm S. et al. Sarcopenic obesity: definition, cause and consequences. Curr Opin Clin Nutr Metab Care, 11 (2008), pp. 693-700.

A downward metabolic spiral nicknamed the “metabaging cycle” describes an aging process influenced by hyperlipidemia and fat deposition into muscle, further worsening sarcopenia. Li C. et al. Pathogenesis of sarcopenia and the relationship with fat mass: descriptive review. J Cachexia Sarcopenia Muscle, 13 (2) (2022), pp. 781-794. <doi: 10.1002/jcsm.12901>.

Excess serum lipids generate an inflammatory cycle, including fat deposition intramuscularly, including an increase in reactive oxygen species, and production of adipokines and inflammatory cytokines. “Inflammaging” then results, causing less fat to be stored, and a relative excess of circulating free fatty acid). Lackey D., Olefsky M. et al. Regulation of metabolism by the innate immune system. Nat Rev Endocrinol., 12 (1) (2016), pp. 15-28.

Kob et al. note that obesity can predispose progenitor cells to differentiate into adipose rather than muscle cells, when given a paracrine signal to do so from inflammatory cytokines. Kob R. et al. Sarcopenic obesity: molecular clues to a better understanding of its pathogenesis? Biogerontology, 16 (2015), pp. 15-29.

Several studies have indicated that obesity can worsen sarcopenia, leading to increased fat infiltration into the muscles, diminished physical function, and a higher mortality risk. Kalinkovich A, Livshits G. (2017). Sarcopenic obesity or obese sarcopenia: A cross talk between age-associated adipose tissue and skeletal muscle inflammation as a main mechanism of pathogenesis. Ageing research reviews. 35, 200-221. http://doi: 10.1016/j.arr.2016.09.008; Barbat-Artigas S., Pion H., Leduc-Gaudet P., Rolland Y., Aubertin-Leheudre M. Exploring the role of muscle mass, obesity, and age in the relationship between muscle quality and physical function. J Am Med Dir Assoc., 15 (4) (2014), pp. 303.e313-320. <doi: 10.1016/j.jamda.2013.12.008>.

Resistance training is recognized as an effective method for mitigating the symptoms of sarcopenia and osteoporosis. Avila J., Gutierres A., Sheehy E., Lofgren E., Delmonico J. (2010). Effect of moderate intensity resistance training during weight loss on body composition and physical performance in overweight older adults. Eur. J. Appl. Physiol., 109 (2010), pp. 517-525.

Nevertheless, the dropout rate from such training programs among older adults can reach as high as 33%. Katz B, Duncan D. Lifting and Toning of Arms and Calves Using High-Intensity Focused Electromagnetic Field (HIFEM) Procedure Documented by Ultrasound Assessment. J Drugs Dermatol, 20 (7) (2021), pp. 755-759.

Increased age and poorer physical performance have been linked to a greater likelihood of discontinuation. Rosado H., Bravo J., Raimundo A., Carvalho J., Marmeleira J., Pereira C. Effects of two 24-week multimodal exercise programs on reaction time, mobility, and dual-task performance in community-dwelling older adults at risk of falling: a randomized controlled trial. BMC Public Health, 10: 21 (Suppl 2) (2021), p. 408. <doi: 10.1186/s12889-021-10448-x>.

Consequently, there is a long felt but unresolved need for alternative approaches to enhance body composition and mitigate muscle and bone loss, as well as total body fat reduction. There would be a substantial advantage in embodiments of a solid composition for oral delivery, or a topical composition for application to an area of the skin, each of the dosage forms effective to reduce visceral fat deposition and/or preserving or enhancing skeletal muscle mass.

### II. SUMMARY OF THE INVENTION

Accordingly, a broad object of particular embodiments of the invention can be provide an admixture of dry ingredients useful in the preparation of solid dosage forms which by oral delivery, or topical dosage forms which by application to the skin, can be effective in the reduction of visceral fat deposition and/or preserving or enhancing skeletal muscle mass. In particular embodiments, the admixture of dry ingredients can comprise, consist essentially of, or consist of, one or more of: Gynostemma Powder Extract, Mulberry Leaf Extract, Goji Berry Powder Extract, Sage Extract, Coffee Robusta Extract, Cardamom Seed Extract, Astragalus Root Extract, and Panax notoginseng Root Extract.

Another broad object of particular embodiments of the invention can be to provide a method of admixing dry ingredients useful in the preparation of solid dosage forms which by oral delivery, or topical dosage forms which by application to the skin, can be effective in the reduction of visceral fat deposition and/or preserving or enhancing skeletal muscle mass, wherein the method comprises, consists essentially, or consists of admixing one or more of: Gynostemma Powder Extract, Mulberry Leaf Extract, Goji Berry Powder Extract, Sage Extract, Coffee Robusta Extract, Cardamom Seed Extract, Astragalus Root Extract, and Panax Notoginseng Root Extract.

Another broad object of particular embodiments of the invention can be to provide a carrier composition adapted to blend with an admixture of dry ingredients to provide a topical composition effective in the reduction of visceral fat deposition and/or preserving or enhancing skeletal muscle mass, wherein the dry ingredients blended with the carrier composition can comprise, consist essentially of, or consist of one or more of: Gynostemma Powder Extract, Mulberry Leaf Extract, Goji Berry Powder Extract, Sage Extract, Coffee Robusta Extract, Cardamom Seed Extract, Astragalus Root Extract, and Panax Notoginseng Root Extract.

Another broad object of the invention can be to provide a topical composition applied to the skin effective to reduce visceral fat deposition and/or preserve or enhance skeletal muscle mass, wherein the topical composition includes one or more dry ingredients comprising, consisting essentially of, or consisting of, one or more of: Gynostemma Powder Extract, Mulberry Leaf Extract, Goji Berry Powder Extract, Sage Extract, Coffee Robusta Extract, Cardamom Seed Extract, Astragalus Root Extract, and Panax Notoginseng Root Extract, blended into a carrier composition.

Another broad object of the invention can be to provide a method of making a topical composition applied to the skin effective to reduce visceral fat deposition and/or preserve or enhance skeletal muscle mass, wherein the topical composition includes one or more dry ingredients comprising, consisting essentially of, or consisting of, one or more of: Gynostemma Powder Extract, Mulberry Leaf Extract, Goji Berry Powder Extract, Sage Extract, Coffee Robusta Extract, Cardamom Seed Extract, Astragalus Root Extract, and Panax Notoginseng Root Extract, blended into a carrier composition.

Naturally, further objects of the invention are disclosed throughout other areas of the specification, drawings, photographs, and claims.

### III. A BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block flow diagram depicting a method of making a particular embodiment of a carrier composition, also referred to as a Phase A carrier composition.

FIG. 2 is a block flow diagram depicting a method of making a particular embodiment of a carrier composition, also referred to as a Phase B carrier composition, and a method of making a particular embodiment of a carrier composition by admixing the Phase A carrier composition with the Phase B carrier composition.

FIG. 3 is a block flow diagram depicting a method of making a particular embodiment of a topical composition, also referred to as a Phase C composition, by combining the Phase A carrier composition with the Phase B carrier composition and blending an admixture of dry ingredients, also referred to as Part C dry ingredients.

FIG. 4 is a bar graph depicting average changes in body biometrics across a study population after a treatment with a particular embodiment of the topical composition for a period of two months, wherein PBF=percent body fat (%); SMM=skeletal muscle mass (lb); VAT=visceral adipose tissue (cm²); and BMR=basal metabolic rate (kcal).

FIG. 5 is a bar graph depicting overall body morphology change evaluation of the study population after treatment with a particular embodiment of the topical composition for a period of two months as graded by three blinded evaluators using a five-point Likert scale.

FIG. 6 includes images of a 39-year-old female before treatment with a particular embodiment of the topical composition (left image) and after a treatment with the particular embodiment of the topical composition for a period of two months (right image).

FIG. 7 includes images of a 41-year-old male before treatment with a particular embodiment of the topical composition (left) and after a treatment with the particular embodiment of the topical composition for a period of two months (right image).

FIG. 8 is a bar graph depicting changes in biometrics for the 41-year-old male depicted in the images of FIG. 7 at one month and two months since start of twice daily application of a particular embodiment of the topical composition, wherein PBF=percent body fat (%); SMM=skeletal muscle mass (lb); VAT=visceral adipose tissue (cm²); and BMR=basal metabolic rate (kcal).

FIG. 9 is a bar graph depicting overall body morphology change evaluation of the 41-year-old male depicted in FIG. 7 after a treatment with the particular embodiment of the topical composition for a period of two months as graded by three blinded evaluators using a five-point Likert scale.

FIG. 10 includes images of a 78-year-old female before treatment with a particular embodiment of the topical composition (left image) and after treatment with the particular embodiment of the topical composition for a period of two months (right image).

FIG. 11 is a bar graph depicting changes in biometrics for the 78-year-old female at one and two months since start of twice daily application of a particular embodiment the topical composition, wherein PBF=percent body fat (%); SMM=skeletal muscle mass (lb); VAT=visceral adipose tissue (cm²); and BMR=basal metabolic rate (kcal).

FIG. 12 is a bar graph depicting overall body morphology change evaluation of the 78-year-old female after a treatment period of two months as graded by three blinded evaluators using a five-point Likert scale.

FIG. 13 includes images of an 80-year-old male before treatment with a particular embodiment of the topical composition (left image) and after a treatment with the particular embodiment of the topical composition for a period of two months (right image).

FIG. 14 is a bar graph depicting changes in biometrics for the 80-year-old male at one and two months since start of twice daily application of a particular embodiment of the topical composition, wherein PBF=percent body fat (%); SMM=skeletal muscle mass (lb); VAT=visceral adipose tissue (cm²); and BMR=basal metabolic rate (kcal).

FIG. 15 is a bar graph depicting average changes in lipid metabolism across a 15 subject study population from inception of treatment with a particular embodiment of the topical composition to the end of a two-month treatment with the particular embodiment of the topical composition.

FIG. 16A is a line graph depicting blood level of alkaline phosphatase across the 15 subject study population from inception of treatment with a particular embodiment of the topical composition to the end of a two-month treatment with the particular embodiment of the topical composition.

FIG. 16B is a line graph depicting blood level of alanine aminotransferase across the 15 subject study population from inception of treatment with a particular embodiment of the topical composition to the end of a two-month treatment with the particular embodiment of the topical composition.

FIG. 16C is a line graph depicting blood level of aspartate aminotransferase across the subject study population from inception of treatment with a particular embodiment of the topical composition to the end of a two-month treatment with the particular embodiment of the topical composition.

### IV. DETAILED DESCRIPTION OF THE PREFERRED EMBODIMENTS

Compositions and methods of preparing and using such compositions to reduce fat deposition and/or preserve or enhance muscle mass. Embodiments of the composition for reduction of fat deposition can comprise, consist essentially of, or consist of an admixture of dry ingredients as described herein or equivalents thereof. In particular embodiments, the composition can comprise, consist essentially of, or consist of an admixture of dry ingredients combined with a carrier composition to aid delivery of the constituents of the admixture of dry ingredients and/or the constituents of the carrier composition. The carrier composition can, as illustrative examples, comprise an ointment, a gel, a lotion, a cream, or other topical carrier adapted to aid delivery of constituents of the admixture of dry ingredients and constituents of the carrier composition, including the active agents therein, to or through the skin by transepidermal route, transappendageal route, or combinations thereof.

In particular embodiments, the delivery system can employ reversible electroporation, in which electrostatic repulsion creates short-lived but high voltage pulses in order to open ionic channels on the skin allowing delivery of constituents of the admixture of dry ingredients and/or constituents of the carrier composition, including the active agents therein, within a topical composition through the skin. Application and delivery of the constituents of the admixture of dry ingredients and/or the constituents of carrier composition within a topical composition can result in a cascade of physiologic triggers that can enhance lipid metabolism.

One target of the active agents in the topical composition can be transcription factor SREBP-1 which regulates the conversion of glucose into fatty acids. Transcription factor SREBP-1 can be inhibited by a polysaccharide included in particular embodiments of the topical composition. Chen M., Xu J., Wang Y., Wang Z., Guo L., Li X., Huang L., Arctium lappa L. The polysaccharide can regulate lipid metabolism in type 2 diabetic rats through the SREBP-1/SCD-1 axis. Carbohydr Res., 494 (2020), p. 108055. <doi: 10.1016/j.carres.2020.108055>. Inhibiting this protein can limit lipogenesis and can reduce, substantially reduce, or prevent long-term energy storage in the form of fat deposition.

Another target of the active agents in the topical composition can be adenosine monophosphate activated protein kinase (“AMPK”) which when stimulated can be an important regulator of cellular metabolism. Li Y., Xu S., Mihaylova M., Zheng B., Hou X., Jiang B., Park O., Luo Z., Lefai E., Shyy Y., Gao B., Wierzbicki M., Verbeuren J., Shaw J., Cohen A., Zang M. AMPK phosphorylates and inhibits SREBP activity to attenuate hepatic steatosis and atherosclerosis in diet-induced insulin-resistant mice. Cell Metab., 13 (4) (2011), pp. 376-388. <doi: 10.1016/j.cmet.2011.03.009>. AMPK, which declines with advancing age, can be found in every cell in the body. It is essential for energy production and utilization of fat for fuel, especially visceral fat. Particular embodiments of the topical composition can act to increase expression of AMPK.

Active agents in particular embodiments of the topical composition can increase expression of ursolic acid which can act to reduce inflammation caused by proinflammatory cytokines released by visceral fat. Katashima K., Silva R., Gomes L., Pichard C., Pimentel D. Ursolic acid and mechanisms of actions on adipose and muscle tissue: a systematic review. Obes Rev., 18 (6) (2017), pp. 700-711. <doi: 10.1111/obr.12523>.

Ursolic acid can also act to increase the body's levels of uncoupling proteins, both UCP-1 and UCP-3. These proteins are responsible for generating thermogenesis in a non-shivering manner within brown adipose tissue. Overexpression of these uncoupling proteins can also transform white adipose tissue (WAT) into beige adipose tissue, which increases utilization of fat tissue for heat and energy generation. Bargut L., Souza-Mello V., Aguila B., Mandarim-de-Lacerda A. Browning of white adipose tissue: lessons from experimental models [online]. Horm Mol Biol Clin Investig. (2017), 31 (1)/j/hmbci.2017.31.issue-1/hmbci-2016-0051/hmbci-2016-0051.xml. [retrieved Jun. 2, 2023]. Retrieved from Internet: <https://pubmed.ncbi.nlm.nih.gov/28099124/><doi: 10.1515/hmbci-2016-0051>.

Illustrative embodiments of an inventive composition comprising, consisting essentially of, or consisting of an admixture of dry ingredients are set forth in the Tables that follow. In particular embodiments, the relationship between the ingredients is further defined as percent by weight (“w/w %”).

In particular embodiments, an inventive admixture of dry ingredients effective to reduce visceral fat deposition and/or preserve or enhance muscle mass can comprise, consist essentially of, or consist of the dry ingredients set forth in Table 1. The relative weight percent can be adjusted based on the dosage form and delivery route.

TABLE 1

| MATERIAL | CAS NUMBER |
|---|---|
| Gynostemma Powder Extract | CAS # 80321-63-7 |
| Sage Extract | CAS # 84082-79-1 |
| Cardamom Powder Extract | CAS # 8000-66-6 |
| Coffee Robusta Extract | CAS # 97593-13-0 |
| Notoginseng Extract | CAS # 94279-78-4 |

In particular embodiments, an inventive admixture of dry ingredients effective to reduce visceral fat deposition and/or preserve or enhance muscle mass can be produced in batches scaled by use of the range of percents by weight set forth in Table 2. The resulting batch can then be divided into dosage amounts of about 0.1 grams to about 0.2 grams. As a further illustrative example, an effective topical composition for the reduction of visceral fat deposition can include about 0.1 grams to about 0.5 grams of the inventive admixture of dry ingredients per milliliter of a topical composition. As a further illustrative example, a particular embodiment of a topical composition of 50 milliliters can include about 5 grams to about 10 grams of the inventive admixture of dry ingredients blended with a carrier composition suitable for application to the skin.

TABLE 2

| MATERIAL | CAS NUMBER | PERCENT BY WEIGHT |
|---|---|---|
| Gynostemma Powder Extract | CAS # 80321-63-7 | about 39.0% to about 43.0% |
| Sage Extract | CAS # 84082-79-1 | about 21.0% to about 25.0 |
| Cardamom Powder Extract | CAS # 8000-66-6 | about 16% to about 20% |
| Coffee Robusta Extract | CAS # 97593-13-0 | about 11.0% to about 15% |
| Notoginseng Extract | CAS # 94279-78-4 | about 3.0% to about 7.0% |

In particular embodiments, an inventive admixture of dry ingredients effective to reduce visceral fat deposition and/or preserve or enhance muscle mass can comprise, consist essentially of, or consist of the dry ingredients set forth in Table 3 with the relative weight percent adjusted based on the dosage form and delivery route.

TABLE 3

| MATERIAL | CAS NUMBER |
|---|---|
| Gynostemma Powder Extract | CAS # 80321-63-7 |
| Mulberry Leaf Extract | CAS # 94167-05-2 |
| Goji Berry Powder Extract | CAS # 85085-46-7 |
| Sage Extract | CAS # 84082-79-1 |
| Coffee Robusta Extract | CAS # 97593-13-0 |
| Cardamom Powder Extract | CAS # 8000-66-6 |
| Astragalus Root Extract | CAS # 94166-93-5 |
| Panax Notoginseng Extract | CAS # 94279-78-4 |

In particular embodiments, an inventive admixture of dry ingredients effective to reduce visceral fat deposition can be produced in batches scaled by use of the range of weight percents set forth in Table 4. The resulting batch can then be divided into dosages of about 0.1 grams to about 0.2 grams each. As a further illustrative example, an effective topical composition for the reduction of visceral fat deposition and/or preserve or enhance muscle mass can include about 0.1 grams to about 0.2 grams of the inventive admixture of dry ingredients per milliliter of the topical composition. As a further illustrative example, a particular embodiment of a topical composition of 50 milliliters can include about 5 grams to about 10 grams of the inventive admixture of dry ingredients blended with a carrier composition suitable for application to the skin. In particular embodiments, the inventive admixture of dry ingredients in the amount of about 5.5 grams to about 6.5 grams can be blended with 50 mL of a carrier composition to produce a topical composition effective upon application to reduce visceral fat deposition and/or preserve or enhance muscle mass.

TABLE 4

| MATERIAL | WEIGHT PERCENT |
|---|---|
| Gynostemma Powder Extract | about 29.0% to about 34.0% |
| Mulberry Leaf Extract | about 16.0% to about 19.0% |
| Goji Berry Powder Extract | about 12.0% to about 16.0% |
| Sage Extract | about 10.0% to about 14% |
| Coffee Robusta Extract | about 8.0% to about 12% |
| Cardamom Seed Extract | about 6.0% to about 10% |
| Astragalus Root Extract | about 2.0% to about 6.0% |
| Panax Notoginseng Root Extract | about 2.0% to about 6.0% |

As an illustrative example, an inventive admixture of dry ingredients effective to reduce visceral fat deposition and/or preserve or enhance muscle mass can be produced in batches scaled by use of the weight percents set forth in Table 5. The resulting batch can then be divided into dosages of about 0.1 grams to about 0.2 grams each. As a further illustrative example, an effective topical composition for the reduction of visceral fat deposition can include about 0.1 grams to about 0.2 grams of the inventive admixture of dry ingredients per milliliter of the topical composition. As a further illustrative example, a particular embodiment of a topical composition of 50 milliliters can include about 5 grams to about 10 grams of the inventive admixture of dry ingredient blended with a carrier composition suitable for application to the skin. In particular embodiments, the inventive admixture of dry ingredients in the amount of about 5.5 grams to about 6.5 grams can be blended with 50 mL of a carrier composition to produce a topical composition effective upon application to reduce visceral fat deposition and/or preserve or enhance muscle mass.

TABLE 5

| MATERIAL | WEIGHT PERCENT |
|---|---|
| Gynostemma Powder Extract | about 31.4% |
| Mulberry Leaf Extract | about 17.7% |
| Goji Berry Powder Extract | about 13.7% |
| Sage Extract | about 11.8% |
| Coffee Robusta Extract | about 9.8% |
| Cardamom Seed Extract | about 7.8% |
| Astragalus Root Extract | about 3.9% |
| Panax Notoginseng Root Extract | about 3.9% |

The ingredients can be further described as follows:

Gynostemma Powder Extract (CAS #80321-63-7): Gynostemma pentaphyllum is a climbing vine found in South and East Asia primarily. It is in the family Cucurbitaceae which includes cucumbers, gourds, and melons. Gynostemma Powder Extract can be an excellent source of saponins, chemicals that the body uses to create several compounds including steroidal hormones. A key saponin in Gynostemma are gypenosides. Gypenosides confer several documented benefits including reducing anxiety, decreasing mast cell production, thereby benefitting allergies, and regulating deposition of body fat. Studies have shown that gypenosides can increase the body's production of AMPK. Choi E. et al. Supplementation with extract of Gynostemma pentaphyllum leaves can reduce anxiety in healthy subjects with chronic psychological stress: A randomized, double-blind, placebo-controlled clinical trial. Phytomedicine. 52 (2019), pp. 198-205. <doi: 10.1016/j.phymed.2018.05.002>; Nguyen P. et al. New dammarane-type glucosides as potential activators of AMP-activated protein kinase (AMPK) from Gynostemma pentaphyllum. Bioorg Med Chem. 19 (21) (2011), pp. 6254-6260. <doi: 10.1016/j.bmc.2011.09.013>.

AMPK, an enzyme found in all cells regulates lipid metabolism and energy production. AMPK declines with advancing age. One of the key actions of AMPK is to trigger burning of stored fat for energy. Anaerobic glycolysis and metabolism, inducing energy conservation, can reduce circulating AMPK, which is a major contributor to abdominal fat formation and storage. Hardie G. AMPK: a key regulator of energy balance in the single cell and the whole organism. Int J Obes (Lond). 32 (Suppl 4) (2008), pp. S7-S12.

Increased AMPK production can support body fat reduction by promoting utilization and oxidation of glucose while decreasing appetite. AMPK can increase fatty acid oxidation (burning fat for energy) while simultaneously reducing fatty acid synthesis. AMP-activated protein kinase [online]. Wikipedia [retrieved Jun. 2, 2023]. Retrieved from Internet: <https://en.wikipedia.org/wiki/AMP-activated_protein kinase>.

Mulberry Leaf Extract (CAS #94167-5-2): Mulberry (Morus alba) is a shrub or tree native to Asia. One of the constituent compounds in the Mulberry Leaf Extract, 1-deoxynojirimycin, inhibits the breakdown of ingested sugars and starches into glucose. This inhibition translates into less insulin output and ultimately into reduced fatty acid synthesis and fat deposition. Mulberry Leaf Extract also contains chlorogenic acid and its related chemicals (isomers), rutin, quercetin, resveratrol, quercetin, p-hydroxycinnamic acid, and caffeic acid. Mulberry Leaf Extract can activate UCP-1, AMPK, PPAR alpha, and PGC 1 alpha. Lim H., Lee O., Kim Y., Yang J, Lim Y. Anti-inflammatory and anti-obesity effects of mulberry leaf and fruit extract on high fat diet-induced obesity. Exp Biol Med. 238 (2013), pp. 1160-1169. <doi: 10.1177/1535370213498982>. Mulberry Leaf Extract can activate brown adipose tissue and can induce browning of inguinal white adipose tissue in type 2 diabetic rats through regulating the AMP-activated protein kinase signaling pathway. Cheng L. et al. Mulberry leaf activates brown adipose tissue and induces browning of inguinal white adipose tissue in type 2 diabetic rats through regulating AMP-activated protein kinase signalling pathway [online]. Br J Nutr. (2022) 127 (6), pp. 810-822 [retrieved Jun. 2, 2023]. Retrieved from Internet: <https://pubmed.ncbi.nlm.nih.gov/33971987/>.

Goji Berry Powder Extract (CAS #85085-46-7): Goji Berry, also known as Wolfberry, is a bright red fruit native to Asia. Goji Berries offer a wide range of nutrients including vitamins, minerals and anti-oxidants. A constituent of Goji Berry Powder Extract comprises a water-soluble polysaccharide fraction (glycoconjugates) clinically demonstrated to have beneficial effects on blood sugar metabolism and body composition. Masci A., Carradori S., Casadei M. A., Paolicelli P., Petralito S., Ragno R., Cesa S. Lycium barbarum polysaccharides: Extraction, purification, structural characterisation and evidence about hypoglycaemic and hypolipidaemic effects. A review. Food Chem. 254 (2018), pp. 254: 377-389. <doi: 10.1016/j.foodchem.2018.01.176>[retrieved Jun. 2, 2023]. Retrieved from Internet: <https://www.sciencedirect.com/journal/food-chemistry>.

Polysaccharides contained in Goji Berry Powder Extract can activate AMPK and suppress sterol regulatory element-binding protein-1c (SREBP-1c). SREBP-1c is a transcription factor required for glucose utilization and fatty acid and lipid production. SREBP-1c activated in the liver by insulin results in insulin output and ultimately produces excessive fat storage. Inhibition of SREB-1c has been clinically shown to reduce fat deposition. Lee W., Lee Y., Wang Q., Yang Y. Crude extracts from Lycium barbarum can suppress SREBP-1c expression and prevent diet-induced fatty liver through AMPK activation. Biomed Res Int. (2014) p. 196198. <doi: 10.1155/2014/196198>; Samar E., Beyleroglu M. The effect of goji berry consumption on weight loss in boxers [online]. Progress in Nutrition 22 (1-S) (2020), pp. 103-110 [retrieved on Jun. 2, 2023]. Retrieved from Internet: <https://www.mattioli1885journals.com/index.php/progressinnutrition/issue/view/750><doi.org/10.23751/pn.v22i1-S.9799>.

Sage Extract (CAS #84082-79-1): Sage is a well-known and commonly used herb for both culinary and medicinal uses. Sage officinalis contains several bioactive compounds including tannins (salviatannin), essential oils including one or more of alpha-thujone, beta-thujone, cineole, camphor, flavones, phenolic acids, and various terpenes. Sage can be a source of ursolic acid which, as above described, can increase the expression of AMPK and irisin in skeletal muscles. This directly leads to improved lipid metabolism and increase in lean tissue synthesis. Seo D. et al. Ursolic acid in health and disease. Korean J Physiol Pharmacol. 22 (3) (2018), pp. 235-248.

Additionally, ursolic acid can increase levels of Uncoupling Protein 1 (UPC1). UPC1 can be a respiration uncoupler responsible for generating non-shivering thermogenesis in brown fat tissue. Upon activation it stimulates the production of heat rather than ATP within brown adipose tissue. It is also thought to occur in beige adipocytes. Ursolic Acid has also been proven to upregulate Uncoupling Protein 3 (UPC 3). Specifically, UCP1 and UCP3 expression affects free fatty acid catabolismas a downstream target of AMPK activation in skeletal muscle. Chu X. et al. Ursolic acid increases energy expenditure through enhancing free fatty acid uptake and β-oxidation via an UCP3/AMPK-dependent pathway in skeletal muscle. Mol Nutr Food Res. 59 (8) (2015), pp. 1491-503. <doi: 10.1002/mnfr.201400670>.

Coffee Robusta Extract (CAS #97593-13-0): Coffee Robusta accounts for about 40% of global coffee production. It is grown in Africa, Indonesia, with the largest producer being Vietnam. Coffee Robusta Extract is derived from unroasted coffee bean to retain heat sensitive compounds. Coffee Robusta Extract can be standardized to 50% chlorogenic acid. Chlorogenic acid can improve cardiovascular health, normalize blood pressure, balance blood sugar, reduce inflammation, and reduce fatty liver disease. Tajik N., Tajik M., Mack I., Enck P. The potential effects of chlorogenic acid, the main phenolic components in coffee, on health: a comprehensive review of the literature. Eur J Nutr. 56 (7) (2017), pp. 2215-2244. <doi: 10.1007/s00394-017-1379-1>. Chlorogenic acid can increase browning of adipocytes by increasing expression of UCP1 and peroxisome proliferator-activated receptor-gamma coactivator 1 alpha (PGC 1A). PGC 1A is a member of a family of transcription coactivators that plays a central role in the regulation of cellular energy metabolism. Sudhakar M. et al. Chlorogenic acid promotes development of brown adipocyte-like phenotype in 3T3-LI adipocytes. [online]. Journal of Functional Foods (74) (2020) [retrieved on Jun. 2, 2023]. Retrieved from Internet: https://www.sciencedirect.com/science/article/pii/S1756464620303856.

PGC 1A can be involved in disorders such as obesity, diabetes, and cardiomyopathy. PGC1A was originally described as a coactivator of PPARy that modulated expression of uncoupling protein 1 (UCP1) and thermogenesis in brown fat. It has also been shown to control mitochondrial biogenesis and oxidative metabolism in many cell types. PGC1A can be induced in muscle by exercise and can stimulate beneficial effects of exercise in muscle.

Cardamom Powder Extract (CAS #8000-66-6): Cardamom is a spice native to the Indian subcontinent and Indonesia. It is commonly used in Indian dishes and is a member of the Ginger family. Cardamom is a rich source of several terpenes including α-terpineol, myrcene and limonene. Clinical studies evidence that ingestion of cardamom can increase serum irisin levels as well as improve fasting blood sugar (FBS), insulin (FBI), total cholesterol (TC), triglyceride (TG), low-density lipoprotein cholesterol (LDL-c), and high-density lipoprotein cholesterol (HDL-c). Daneshi-Maskooni M. et al. Green cardamom supplementation improves serum irisin, glucose indices, and lipid profiles in overweight or obese non-alcoholic fatty liver disease patients: a double-blind randomized placebo-controlled clinical trial. BMC Complement Altern Med. 19 (1) (2019), p. 59. <doi: 10.1186/s12906-019-2465-0>. Even moderately increased levels of circulating irisin can increase energy expenditure, reduce body weight and improves diet-induced insulin resistance. Zhang Y. et al. Irisin stimulates browning of white adipocytes through mitogen-activated protein kinase p38 MAP kinase and ERK MAP kinase signaling. Diabetes, 63 (2) (2014), pp. 514-525. <doi: 10.2337/db13-1106>.

Astragalus Root Extract (CAS #94166-93-5): Astragalus is a genus comprised of over 3000 herbs and shrubs. It is in the legume family and grown in climates in the northern hemisphere. It has been used for centuries in and considered one of the most useful herbs in traditional Chinese medicine. Astragalus's constituents include astragalosides. Astragalosides are terpene glycoside compounds that have a wide range of action including anti-oxidant, anti-cancer, immune enhancement, improved digestion, increased energy while reducing fatigue, and can also be used to protect cellular integrity during chemotherapy.

Recent evidence shows that Astragalus can improve leptin sensitivity. Leptin is essential for appetite control, but leptin resistance is a common problem with overweight individuals. Consequently, reestablishing leptin sensitivity can act to reduce excess weight, and adipose tissue. Astragalus root can increase the transport of leptin across the blood brain barrier, thereby inhibiting obesity. Blood leptin concentration can be up to three times higher in people with obesity than in lean people, but the blood leptin concentration in the cerebrospinal fluid of people with obesity can be only 30% of that of lean people. Astragalus also helps to reduce inflammation, a major contributing factor to visceral fat chronicity. Shao J. et al. Recent evidence in support of traditional Chinese medicine to restore normal leptin function in simple obesity [online]. Heliyon (2022 May 19 [retrieved Jun. 2, 2023]. Retrieved from on Internet: <https://pubmed.ncbi.nlm.nih.gov/35620623/><doi: 10.1016/j.heliyon.2022.e09482>. Another component of Astragalus is formononetin. Formononetin can upregulate UCP1 and PPARy. Formononetin regulates adipocyte thermogenesis as a non-classical PPARy agonist. Nie T. et al. The natural compound, formononetin, extracted from Astragalus membranaceus increases adipocyte thermogenesis by modulating PPARγ activity. Br J Pharmacol. 175 (9) (2018), pp. 1439-1450. <doi: 10.1111/bph.14139>.

Panax Notoginseng Extract (CAS #94279-78-4): Panax Notoginseng is a species of Panax that grows naturally in China. It is also known as Chinese or Tienchi Ginseng. Panax Notoginseng contains several dammarane type ginsenosides. Zhang X. et al. Effect of Panax notoginseng Saponins and Major Anti-Obesity Components on Weight Loss [online]. Pharmacol. 11 (2020), p. 601751 [retrieved on Jun. 2, 2023]. Retrieved from Internet: <https://www.frontiersin.org/articles/10.3389/fphar.2020.601751/full><doi: 10.3389/fphar.2020.601751>. Dammarane type ginsenosides are tetracyclic triterpenes and form triterpene saponins such as those found in certain Ginsengs. Dammaranes are known for improving energy, immunity, lowering lipid levels and blood pressure, reducing osteoporosis, protecting the liver, and increasing longevity. Dammaranes are also neuroprotective, cardioprotective, and have exhibited anti-cancer potential. Yoshikawa M. et al. Bioactive saponins and glycosides. XIX. Notoginseng (3): immunological adjuvant activity of notoginsenosides and related saponins: structures of notoginsenosides-L, -M, and -N from the roots of Panax notoginseng. Chem Pharm Bull (Tokyo) 49 (11) (2001), pp. 1452-1456. <doi: 10.1248/cpb.49.1452>. Notoginseng can affect weight through a number of pathways. The ginsenosides impact UCP1 and AMPK which help to increase the browning of white adipose tissue. Notoginseng can also improve glucose metabolism by stimulating Insulin Receptor Substrate-1 (IRS1) to increase insulin sensitivity, and improve skeletal muscle glucose uptake through its effect on Glucose transporter 4 (GLUT4). T. C. Chang, et al. Effect of ginsenosides on glucose uptake in human Caco-2 Cells is mediated through altered Na+/glucose cotransporter 1 expression. Journal of Agricultural Food Chemistry 55 (2007), pp. 1993-1998. Additional benefits include upregulating AMPK, PGC 1A and Sirtuin1 (SIRT1). SIRT1 is a cellular energy sensor involved in glucose and lipid metabolism, browning of white fat, and mitochondrial biogenesis. Y. C. Huang, et al. Effect and mechanism of ginsenosides CK and Rgl on stimulation of glucose uptake in 3T3-L1 Adipocytes. Journal of Agricultural Food Chemistry 58 (2010), pp. 6039-6047. <doi: 10.1021/jf9034755>; Y. C. Huang, et al. An essential role of cAMP response element binding protein in ginsenoside Rg1-mediated inhibition of Na/glucose cotransporter 1 gene expression. Molecular Pharmacology 88 (6) (2015), pp. 1072-83. <doi: 10.1124/mol.114.097352>; W. L. Chang, et al. The inhibitory effect of ginsenoside Rgl on glucose and lipid production in human HepG2 Cells. Adaptive Medicine 5 (4) (2013), pp. 181-188. <doi: 10.4247/AM.2013.ABD068>.

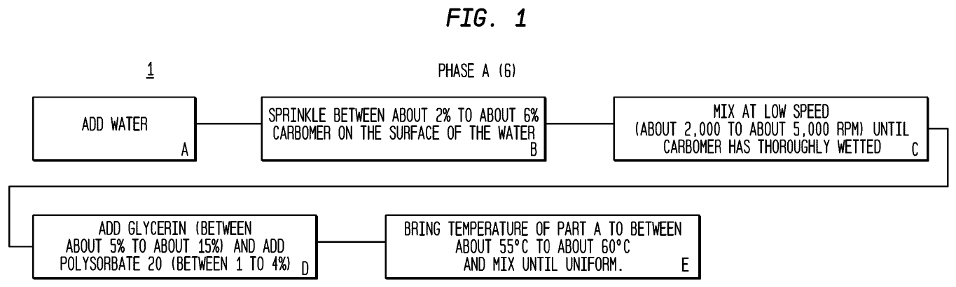

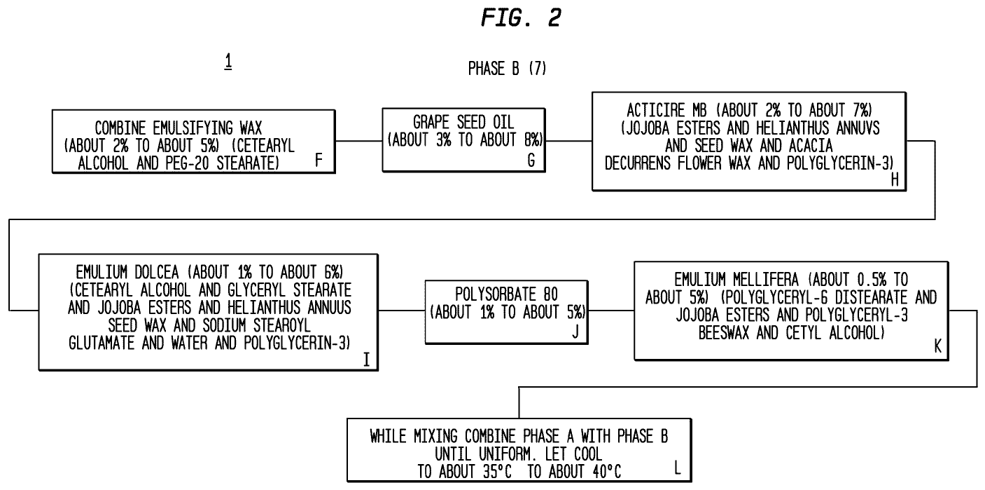

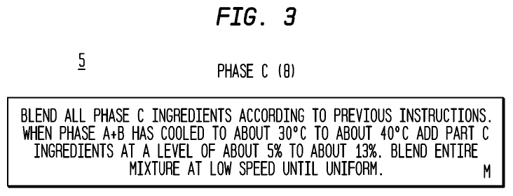

Now with primary reference to FIGS. 1 through 3, illustrating particular embodiments of a carrier composition (1) and methods of making a carrier composition comprising, consisting essentially of, or consisting of an admixture of one or more of Phase A ingredients (2) (as shown in the illustrative examples of Tables 6 through 7 and described within Blocks A through E of the block flow diagram of FIG. 1), or an admixture of one or more of Phase B ingredients (3) (as shown in the illustrative example of Table 8 and described within Blocks F through L of the block flow diagram of FIG. 2), or an admixture of one or more of Phase A ingredients (2) and one or more of Phase B ingredients (3) (as shown in the illustrative examples of FIGS. 1 and 2), and illustrating particular embodiments of a topical composition (5) and methods of making a topical composition (5) comprising, consisting essentially of, or consisting of Phase C ingredients (4) (as shown in the illustrative examples of Tables 1 through 5 and described within Bock M of FIG. 3) blended with a carrier composition (1) which topical composition (5) can be applied to the skin for effective reduction in visceral fat deposition and/or to preserve or enhance muscle mass.

### The Carrier Composition.

Now, with primary reference to FIGS. 1 and 2, illustrating particular embodiments of a carrier composition (1) and methods of making and using particular embodiments of a carrier compositions (1). Illustrative examples of the carrier composition (1) can comprise, consist essentially of, or consist of a Phase A carrier composition (6) including one or more Phase A ingredients (2) and/or comprise, consist essentially of, or consist of a Phase B carrier composition (7) including one or more Phase B ingredients (3). In particular embodiments, each of Phase A carrier composition (6) and Phase B carrier composition (7) can be prepared separate from the other as discrete inventive formulations, while in other embodiments the Phase A carrier composition (6) and Phase B carrier composition (7) can be prepared separately and then subsequently combined to produce embodiments of inventive carrier compositions (1). In particular embodiments the Phase A carrier composition (6) and/or Phase B carrier composition (7) can be combined with a Phase C (8) including an admixture of one or more Phase C ingredients (4) to produce embodiments of a topical compositions (5) which can be applied to the skin to effect reduction of fat deposition and/or preserve or enhance muscle mass.

Phase A Carrier Composition. Now, with primary reference to FIG. 1, the Phase A carrier composition (6) can comprise, consist essentially of, or consist of, one or more of the following Phase A ingredients (2) set forth in Table 6.

TABLE 6
MATERIAL

Water (CAS: # 7732-18-5)
Carbomer (CAS # 76050-42-5)
Glycerin (CAS # 56-81-5)
Polysorbate 20 (CAS # 9005-64-5)

Illustrative embodiments of the Phase A carrier composition (6) can be produced by varying the weight percent (“w/w %”) of each ingredient within the specified range relative to the weight of water as set forth in Table 7.

TABLE 7

| MATERIAL | WEIGHT PERCENT |
|---|---|
| Carbomer (CAS # 76050-42-5) | about 2% to about 6% |
| Glycerin (CAS # 56-81-5) | about 5% to about 15% |
| Polysorbate 20 (CAS # 9005-64-5) | about 1% to about 4% |
| Water (CAS: # 7732-18-5) | the remaining weight percent. |

The Phase A ingredients (2) can be further described as follows:

Water (CAS #7732-18-5) is an ACS Reagent Grade Water which meets ASTM Type I and/or ASTM Type II specifications and may otherwise be referred to as purified water, ultratrace water, HPLC grade water, deionized water, injection quality water, sterile purified water, molecular biology grade water, or equivalents thereof.

Carbomer (CAS #76050-42-5) is poly(acrylic acid) a white powder that is a high molecular weight polymer cross-linked by acrylic acid and allyl sucrose ether or allyl pentaerythritol ether, or equivalents thereof. Carbomer 940 can be used in embodiments as a thickening, suspending, and emulsifying agent.

Polysorbate 20 (CAS #9005-64-5) is 2-[2-[3,4-bis(2-hydroxyethoxy)oxolan-2-yl]-2-(2-hydroxyethoxy) ethoxy]ethyldodecanoate a non-toxic, nonionic surfactant/emulsifier and dispersing agent which can be used in embodiments to allow oil and water to mix without the use of alcohol.

Now, with primary reference to FIG. 1, an illustrative example of a Phase A carrier composition (6) including one or more of the Phase A ingredients (2), set out in Tables 6 and 7, can be produced by a method comprising one or more of: transfer of a measured amount of water (CAS: #7732-18-5) to a mixing vessel of a mixing device (Block A). Sprinkle Carbomer (CAS #76050-42-5) onto the surface of the water, and in particular embodiments about 2% to about 6% (w/w %) (Block B). Mix the carbomer and water admixture at low speed (about 2,000 rpm to about 5,000 rpm) until the carbomer is thoroughly wetted (Block C). Add glycerin (CAS #56-81-5), and in particular embodiments about 5% to about 15% (w/w %), and polysorbate 20 (CAS #9005-64-5), and in particular embodiments about 1% to about 4% (Block D). Bring the temperature of the admixture to about 55° C. to about 60° C. and mix until uniform (Block E).

Phase B Carrier Composition. Now, with primary reference to FIG. 2, Phase B carrier compositions (7) can comprise, consist essentially of, or consist of, one or more of the following Phase B ingredients (3) set forth in Table 8.

TABLE 8
MATERIAL

Emulsifying Wax:
Cetearyl Alcohol (CAS # 67762-27-0)
PEG-20 Stearate (CAS # 9004-99-3)
Grape Seed oil (CAS # 8024-22-4)
Acticire ® MB:
Jojoba Esters (CAS # 85186-93-2)
Helianthus Annuus Seed Wax (CAS #68937-99-5)
Acacia Decurrens Flower Wax (CAS # 98903-76-5)
Polyglycerin-3 (CAS # 56090-54-1)
Emulium ® Dolcea:
Cetearyl Alcohol (CAS # 36653-82-4)
Glyceryl Stearate (CAS # 31566-31-1)
Jojoba Esters (CAS # 85186-93-2)
Helianthus Annuus Seed Wax (CAS # 68937-99-5)
Sodium Stearoyl Glutamate (CAS # 38517-23-6)
Water (CAS # 7732-18-5)
Polyglycerin-3 (CAS # 56090-54-1)
Polysorbate 80 (CAS # 9005-65-6)
Emulium ® Mellifera:
Polyglyceryl-6 Distearate (CAS # 34424-97-0)
Jojoba Esters (CAS # 85186-93-2)
Polyglyceryl-3 Beeswax (CAS # 136097-93-3)
Cetyl Alcohol (CAS # 36653-82-4)

Particular illustrative embodiments of the Phase B carrier composition (7) can be produced by varying the weight percent (“w/w %”) of each Phase B ingredient (3) within the specified range as set forth in Table 9.

TABLE 9

| MATERIAL | WEIGHT PERCENT |
|---|---|
| Emulsifying Wax | about 2% to about 5% |
| Cetearyl Alcohol (CAS # 67762-27-0) | |
| PEG-20 Stearate (CAS # 9004-99-3) | |
| Grape Seed oil (CAS # 8024-22-4) | about 3% to about 8% |
| Acticire ® MB | about 2% to about 7% |
| Jojoba Esters (CAS # 85186-93-2) | |
| Helianthus Annuus Seed Wax (CAS # 68937-99-5) | |
| Acacia Decurrens Flower Wax (CAS # 98903-76-5) | |
| Polyglycerin-3 (CAS # 56090-54-1) | |
| Emulium ® Dolcea | about 1% to about 6% |
| Cetearyl Alcohol (CAS # 36653-82-4) | |
| Glyceryl Stearate (CAS # 31566-31-1) | |
| Jojoba Esters (CAS # 85186-93-2) | |
| Helianthus Annuus Seed Wax (CAS # 68937-99-5) | |
| Sodium Stearoyl Glutamate (CAS # 38517-23-6) | |
| Water (CAS # 7732-18-5) | |
| Polyglycerin-3 (CAS # 56090-54-1) | |
| Polysorbate 80 (CAS # 9005-65-6) | about 1% to about 5% |
| Emulium ® Mellifera | about 0.5% to about 5% |
| Polyglyceryl-6 Distearate (CAS # 34424-97-0) | |
| Jojoba Esters (CAS # 85186-93-2) | |
| Polyglyceryl-3 Beeswax (CAS # 136097-93-3) | |
| Cetyl Alcohol (CAS # 36653-82-4) | |

The ingredients can be further described as follows:

### Emulsifying Wax:

Cetearyl Alcohol (CAS #67762-27-0) is Cetostearyl Alcohol (CH3(CH2)nOH) a mixture of cetyl and stearyl alcohols or equivalents thereof which can function as an emulsion stabilizer, opacifying agent; surfactant-foam booster, and/or viscosity increasing agent.

PEG-20 Stearate (CAS #9004-99-3) is polyethylene glycol C₂ₙH₄ₙ₊₂Oₙ₊₁ or equivalent thereof which can be useful as one or more of a penetration enhancer, surfactant, emulsifier, or humectant.

Grape Seed oil (CAS #8024-22-4) is an oil pressed from seeds of Vitis vinifera, or an equivalent thereof.

### Acticire® MB:

Jojoba Esters (CAS #85186-93-2) is hydrolyzed jojoba esters produced as hydrogenation or transesterification product of Jojoba oil, or equivalents thereof.

Helianthus Annuus Seed Wax (CAS #68937-99-5) is the residue from the expression of oil from the seedcake of sunflower.

Acacia decurrens Flower Wax (CAS #98903-76-5) is the flower wax from the flowers of Acacia decurrens.

Polyglycerin-3 (CAS #56090-54-1) is triglycerol C₉H₂₀O₇ or equivalents thereof.

### Emulium® Dolcea:

Cetyl Alcohol (CAS #36653-82-4) is cetyl alcohol formula CH₃ (CH₂)₁₅OH or equivalent thereof.

Glyceryl Stearate (CAS #31566-31-1) is glyceryl monostearate C₂₁H₄₂O₄ or equivalent thereof.

Jojoba Esters (CAS #85186-93-2) is hydrolyzed jojoba esters produced as hydrogenation or transesterification product of Jojoba oil.

Helianthus Annuus Seed Wax (CAS #68937-99-5) is the residue from the expression of oil from the seedcake of sunflower.

Sodium Stearoyl Glutamate (CAS #38517-23-6) is sodium (4S)-5-hydroxy-4-(octadecanoylamino)-5-oxopentanoate or equivalent thereof.

Water (CAS #7732-18-5) is ACS Reagent Grade Water which meet ASTM Type I or ASTM Type II specifications and may otherwise be referred to as purified water, ultratrace water, HPLC grade water, deionized water, injection quality water, sterile purified water, molecular biology grade water.

Polyglycerin-3 (CAS #56090-54-1) is triglycerol C₉H₂₀O₇.

Polysorbate 80 (CAS #9005-65-6) is polyoxyethylene sorbitan monooleate a nonionic surfactant and emulsifier.

### Emulium® Mellifera:

Polyglyceryl-6 Distearate (CAS #34424-97-0) means distearic acid, diester with hexaglycerol C₅₄H₁₀₆O₁₅ or equivalent thereof.

Jojoba Esters (CAS #85186-93-2) means hydrolyzed jojoba esters produced as hydrogenation or transesterification product of Jojoba oil.

Polyglyceryl-3 Beeswax (CAS #136097-93-3) means a polar beeswax derivative in which the free fatty acids of beeswax have been converted to polyglycerol esters.

Cetyl Alcohol (CAS #36653-82-4) means cetyl alcohol formula CH₃(CH₂)₁₅OH, or equivalent thereof.

Now, with primary reference to FIG. 2, the illustrative example of the Phase B carrier composition (7) including one or more of the Phase B ingredients (3) set out in Tables 8 and 9 can be produced by a method comprising one or more of: admixing (w/w %) in a mixing vessel of a mixing device the emulsifying wax (cetearyl alcohol and PEG-20 stearate) (in particular embodiments emulsifying wax of about 2% to about 5%) (Block F) with grape seed oil (in particular embodiments grape seed oil of about 3% to about 5%) (as shown in Block G), Acticire® MB (Jojoba Esters, Helianthus annuus seed wax, Acacia Decurrens flower wax, Acacia decurrens flower wax, and polyglycerin-3) (in particular embodiments Acticire® MB of about 2% to about 7%) (Block H), Emulium® Dolcea (Cetyl Alcohol, Glyceryl Stearate, Jojoba Esters Helianthus annuus Seed Wax, Sodium Stearoyl Glutamate, water, Polyglycerin-3) (and in a particular embodiments Emulium® Dolcea of about 1% to about 6%) (Block I), Polysorbate 80 (and in particular embodiments Polysorbate 80 of about 1% to about 5%) (Block J), Emulium® Mellifera (Polyglyceryl-6 Distearate, Jojoba Esters, Polyglyceryl-3 Beeswax, Cetyl Alcohol) (and in particular embodiments Emulium® Mellifera of about 0.5% to about 5%) (Block K). Mix until uniform.

Again, with primary reference to FIG. 2, the invention can include embodiments in which the Phase A carrier composition (6) can be admixed to Phase B carrier composition (7) until uniform (as shown by the illustrative example of Block L). In particular embodiments the mixture of Phase A carrier composition (6) and Phase B carrier composition (7) can be cooled to room temperature.

Now, with primary reference to FIG. 3, in particular embodiments, the method can further include a Phase C composition (8) in which the mixture of Phase A (6) and Phase B (7) can be cooled to about 35° C. to about 45° C. (Block L) and Phase C ingredient(s) (4) can be admixed into the combination of Phase A (2) combined with Phase B (7) and mixed at low speed (about 2000 rpm to about 5,000 rpm) until obtaining a uniform mixture of Phase A ingredients (2), Phase B ingredients (3), and Phase C ingredients (4) (Block M) to produce a topical composition (5) containing Phase C ingredients (4) within the range of weight percent set forth in Table 10.

TABLE 10

| MATERIAL | WEIGHT PERCENT |
|---|---|
| Gynostemma Powder Extract | about 0.5% to about 2.5% |
| Mulberry Leaf Extract | about 0.1% to about 1.0% |
| Goji Berry Powder Extract | about 0.1% to about 0.8% |
| Sage Extract | about 0.3% to about 1.5% |
| Coffee Robusta Extract | about 0.2% to about 1.1% |
| Cardamom Seed Extract | about 0.4% to about 1.5% |
| Astragalus Root Extract | about 0.1% to about 0.8% |
| Panax Notoginseng Root Extract | about 0.1% to about 0.8% |

A further illustrative embodiment of the topical composition (5) includes Phase C ingredients (4) by weight percent in a carrier composition (1) as set forth in Table 11, which can, but need not necessarily be, the Phase A carrier composition (6), the Phase B carrier composition (7), or the combination thereof, and in particular embodiments, the carrier composition (6) can comprise can be an equivalent to the Phase A carrier composition (6), the Phase B carrier composition (7), or the combination thereof, or another carrier composition (1) capable of or adapted to blend with Phase C ingredients and suitable for application to the skin.

TABLE 11

| MATERIAL | WEIGHT PERCENT |
|---|---|
| Gynostemma Powder Extract | about 0.5% to about 2.5% |
| Mulberry Leaf Extract | about 0.1% to about 1.0% |
| Goji Berry Powder Extract | about 0.1% to about 0.8% |
| Sage Extract | about 0.3% to about 1.5% |
| Coffee Robusta Extract | about 0.2% to about 1.1% |
| Cardamom Seed Extract | about 0.4% to about 1.5% |
| Astragalus Root Extract | about 0.1% to about 0.8% |
| Panax Notoginseng Root Extract | about 0.1% to about 0.8% |

An embodiment of the inventive topical composition (5) obtained by the method of illustrated by FIGS. 1 through 3 was employed in a study to evaluate efficacy of the topical composition (5) to reduce visceral fat deposition and/or preserve or enhance muscle mass.

The Study. Eligible subjects (21-80 yrs., BMI<33 kg/m²) were included in a study after reviewing inclusion and exclusion criteria. Study subjects accepted into the study expressed a desire to improve their body composition, with willingness to apply the inventive topical composition (5) for transcutaneous delivery twice a day. Exclusion criteria were cardiac disorders requiring medication or treatment, immunosuppressive diseases, poorly controlled endocrine disorders, recent abdominal surgeries, pregnancy, breast feeding, and the use of weight loss medications or testosterone. Fifteen eligible study subjects (9 women and 6 men, 30 to 80 years old, BMI 21 to 33 kg/m²) were enrolled in the study. All participants received treatment instructions and provided written informed consent.

The treatment protocol included application of three pumps of the topical composition (5) (about 3 milliliters to about 5 milliliters) to the abdominal skin twice daily, once in the morning and again at night. Study subjects were strictly instructed to maintain their regular routine diet and exercise program, and to avoid weight gain or loss of over 5 lb from baseline for the study duration. Evaluation parameters included 2D and 3D photographs that were taken in a standardized studio setting before treatment with the topical composition (5), and again at one month, and again two months following treatment inception with the topical composition (5).

The gold standard for biometric analysis is the dual-energy X-ray absorptiometry (DEXA). HF-BIA, such as InBody®, has been shown to be highly correlated with the DEXA for assessing skeletal muscle mass (standard coefficient beta (β)≥0.95) and percent body fat (β≥0.94, R2 ≥0.89). Jensky-Squires E. et al. (2008). Validity and reliability of body composition analysers in children and adults. Br J Nutr. 100 (4), 859-65. https://doi: 10.1017/S0007114508925460. Epub 2008-3-18. PMID: 18346304. Other biometric indices available through this device include weight, BMI, visceral adipose area in cm2, and calculated basal metabolic rate. Basal metabolic rate (BMR) is the amount of energy expended during rest in a neutral environment over a 24-hour period. The InBody® 770 utilizes John J. Cunningham's equation which uses lean body mass (LBM, kg) to estimate BMR (43):

$$BMR ( kcal ) = 21.6 \times LBM + 370$$

The factors in determining BMR among body composition measures are lean body mass, body fat mass, levels of physical activity, and nutrition are also significant determinants. Konarzewski M., Książek A. (2013). Determinants of intra-specific variation in basal metabolic rate. J Comp Physiol B. 183 (1), 27-41. https://doi: 10.1007/s00360-012-0698-z. Epub 2012-7-31. PMID: 22847501; PMCID: PMC3536993. As SMM is a component of LBM, it can be expected that an increase in BMR is correlated with increased SMM and decreased PBF. The transcutaneous topical composition (5) tested showed improvement in study subjects SMM and BMR in conjunction with reduced PBF and VAT following treatment.

HF-BIA, such as InBody®, has been shown to be highly correlated with the DEXA for assessing skeletal muscle mass (standard coefficient beta (B) ≥0.95) and percent body fat (β≥0.94, R2 ≥0.89) (15). Thus, the InBody® 770 was deemed an appropriate instrument to measure body biometrics for this study. Biometric index measurements were taken at the same time points using the InBody® 770, including weight (lb), percent body fat (PBF, %), skeletal muscle mass (SMM, 1b), visceral fat area (VAT, cm²), and basal metabolic rate (BMR, kcal). Fasting liver enzyme panel and lipid panels were drawn from each study subject prior to the start of the study, and again at one month and 2 months post inception.

The primary outcomes and evaluation method assessed body biometrics including weight (lb), percent body fat (PBF, %), skeletal muscle mass (SMM, lb), visceral adipose tissue (VAT, cm²), and (BMR, kcal). The InBody® 770 (Cerritos, CA, USA) was used to measure the changes of these variables, and standardized photographs were taken at baseline, at 1-month, and 2-month follow-up visits. Additionally, as a secondary primary evaluation, blind reviewers assessed study subjects aesthetic improvement including overall abdominal contour, degree of abdominal protuberance, periumbilical skin laxity, and apparent muscle tone using the 5-point Likert scale. The statistical significance was tested by the RStudio and MS Excel utilizing the paired sample t-test with the significance level, a, set at 5% and the null hypothesis set to 0.

The secondary outcomes focused on the complete metabolic panel blood values, specifically the lipid profile and liver metabolism values. Parameters such as triglycerides, cholesterol, HDL, LDL, alanine aminotransferase, alkaline phosphatase, and aspartate aminotransferase were assessed through blood tests. The liver function tests were conducted to ensure the safety of the topical composition (5) regarding its impact on liver function. And lipid metabolism. Measurements were taken at baseline, 1-month, and 2-month follow-up visits.

All fifteen study subjects completed the treatment regime and attended follow-up appointments. No adverse events or significant expected sequelae were reported. Participants did not experience any discomfort or difficulties during the at-home application of the topical composition (5) and were able to resume their daily activities after treatment. The weight of the subjects did not significantly change throughout the study, as their weight remained within a 51b range of baseline (p-value>0.05).

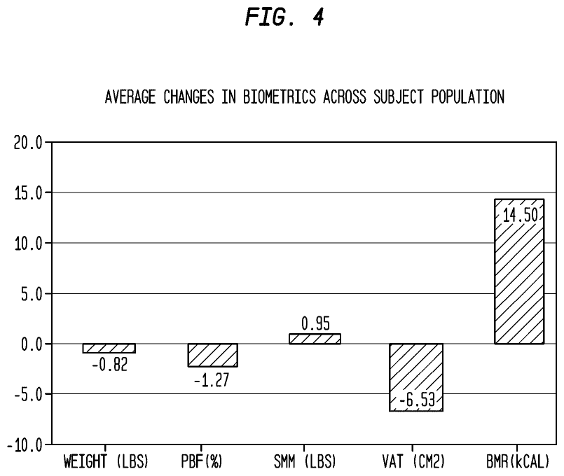

Now, with primary reference to FIG. 4, biometric analysis showed that at baseline, mean weight measured 162.96 lb. The average percent body fat was 29.22%. Mean skeletal muscle mass (SMM) measured 64.42 lb. Mean visceral adipose tissue (VAT) value was 110.03 cm², with a normal value of <100 cm². Basal metabolic rate (BMR) averaged 1519.70 kcal. At the conclusion of the study, the average PBF was lowered to 27.95% (p-value<0.05), a decrease of 1.27%, which translates to a 4.2% lowering of this biometric index. SMM increased to 65.37 lb (p-value<0.05), an increase of 0.95 pounds, without any change in diet or exercise regimes. VAT decreased to 103.51 cm (p-value<0.05), a 5.9% improvement. There was an increase in the average BMR by 14.42 kcal (p-value<0.05). Average weight at baseline was 162.96 lb, and the mean weight decreased slightly to 162.14 lb at the conclusion of the study with a change of −0.82 lb (p-value>0.05).

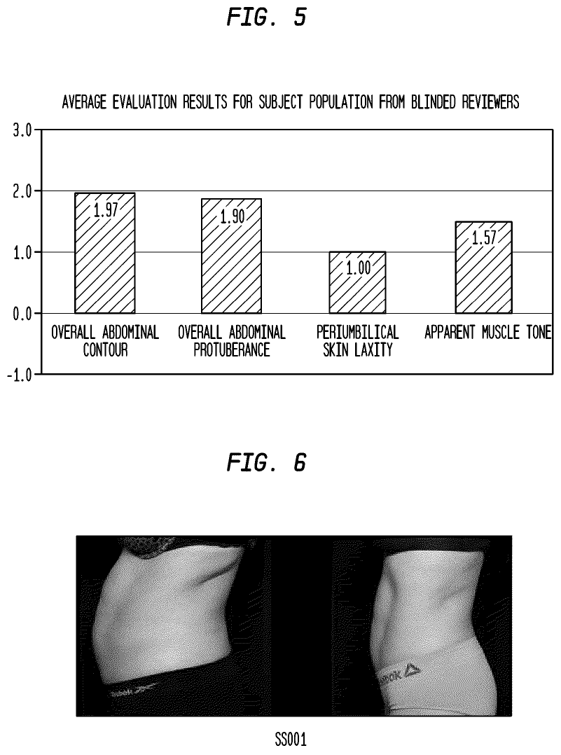

Now, with primary reference to FIG. 5, overall, blinded reviewers evaluating the body morphology improvement in the study subjects observed positive scores for improvement across all categories. No ratings were given by the reviewers to indicate any worsening of appearance.

### ILLUSTRATIVE EXAMPLES

### Example 1

Now, with reference to FIG. 6, depicting a 39-year-old female following childbirth and four pregnancies before treatment (left image), and two months following twice daily application of the topical composition (5) (right image), there was a noted improvement in abdominal contour.

### Example 2

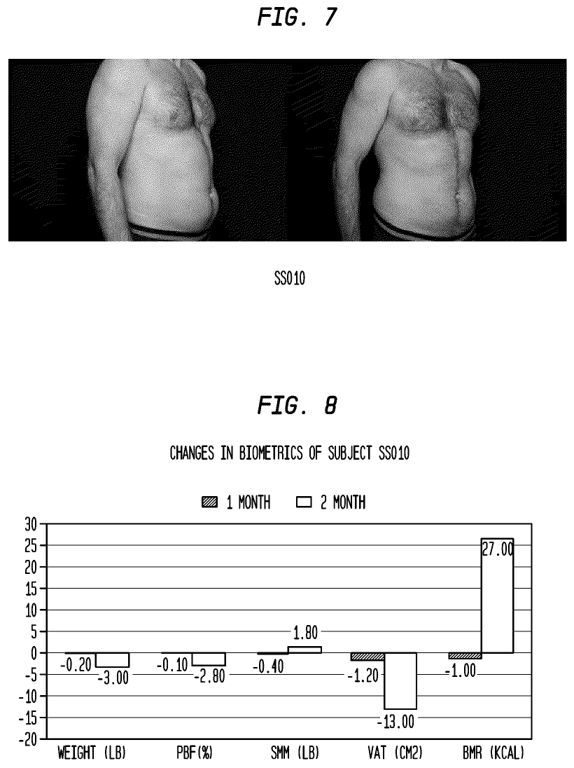

Now, with reference to FIG. 7, depicting a 41-year-old male (subject SS010) with central abdominal protuberance before treatment (left image) and following twice daily application of the topical composition (right image), the epigastric and periumbilical region showed significant improvement in both abdominal protuberance and apparent muscle tone.

Now, with primary reference to FIG. 8, which illustrates the improvement in biometric measurement index changes of subject SS010. PBF decreased by 2.80 percent, with a loss of VAT by 13.00 cm² (−16.31%). SMM improved by 1.80 lb (+2.11%) and achieved a 27.00 kcal improvement in BMR and lost 3.00 lb.

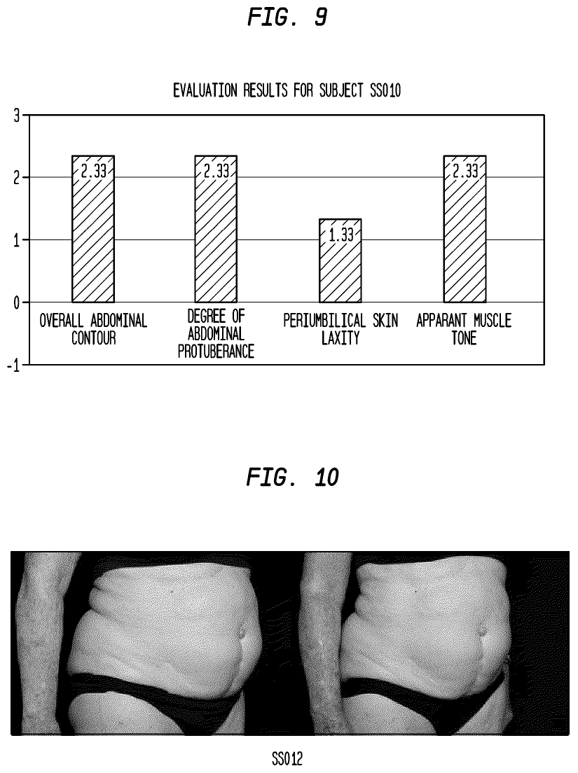

Now, with primary reference to FIG. 9, subject SS010 images shown in FIG. 7 were evaluated by three blinded reviewers. The assessment shows an aesthetic improvement over all categories.

### Example 3

Now, with primary reference to FIG. 10, depicting a 78-year-old-female (SS012) before treatment (left image), and two months following twice daily application of topical composition (right image). The subject body morphology type and age group are clinically difficult to improve.

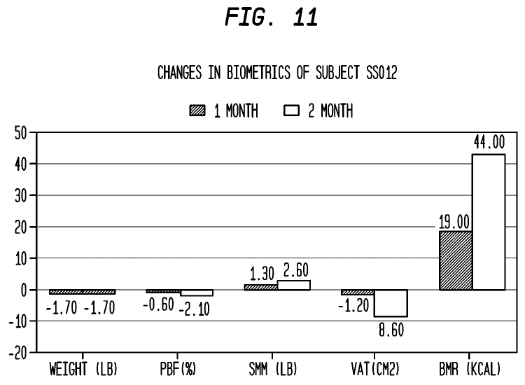

Now, with primary reference to FIG. 11, SS012 body biometrics showed improvement after two months with a decrease in PBF by 2.10%, reduction in VAT by 8.60 cm² (−6.88%), an increase in SMM by 2.60 lb (+3.49%), and a 44.00 kcal improvement in BMR. SS012 weight stayed within 2.0 lb (p-value>0.05) of baseline.

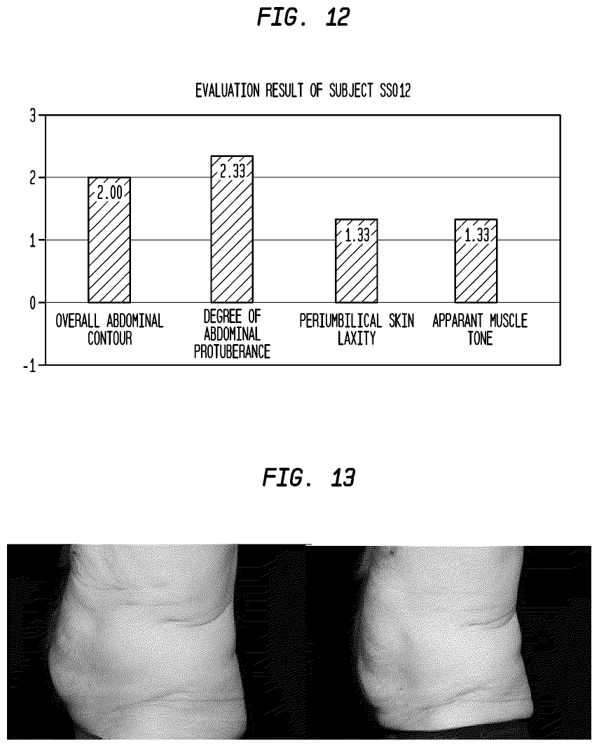

Now, with primary reference to FIG. 12, SS012 images were evaluated by three blinded reviewers. The assessment shows an aesthetic improvement over all categories.

### Example 4

Now, with primary reference to FIG. 13, depicting an 80-year-old male (SS016) before treatment (left image), and two months following twice daily application of topical composition (right image).

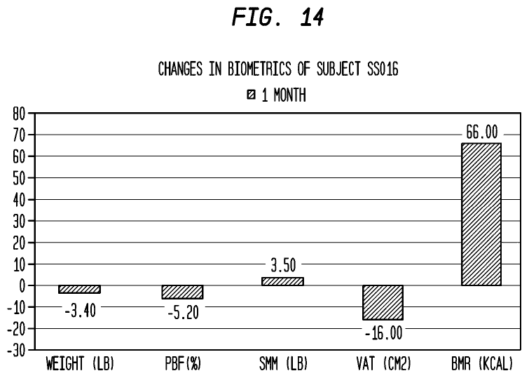

Now, with primary reference to FIG. 14, subject SS016 had significant reduction of visceral fat. Biometric indices showed improvement with a decrease in PBF by 5.20%, reduction in VAT by 16.00 cm² (−16.04%), an increase in SMM by 3.40 lb (+4.77%), and a 66.00 kcal improvement (−3.95%) in BMR. SS016 weight stayed within the required 5.0 lb weight change limit required by the study (p-value>0.05) of baseline.

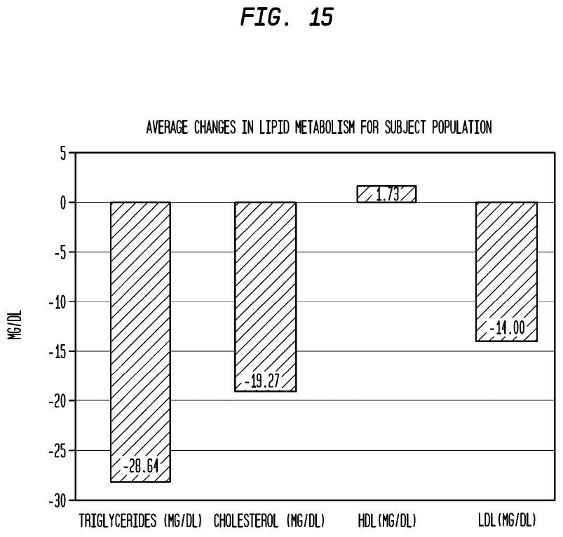

Now, with reference to FIG. 15, overall, of the 15 subject population treated in the study, the majority showed reduction in triglycerides while the others remained within their pre-treatment normal limits. There was an average decrease in triglycerides of 28.64 mg/dL across the patient population (p-value<0.05). There was also a decrease in average cholesterol and LDL across the patient population by 19.27 mg/dL (p-value<0.05) and 14.00 mg/dL (p-value<0.05), respectively. HDL values increased slightly but stayed within the normal range of 35.0 to 65.0 mg/dL for men, 35.0 to 80.0 mg/dL for women. Results of the lipid metabolism evaluation are portrayed in FIG. 12.

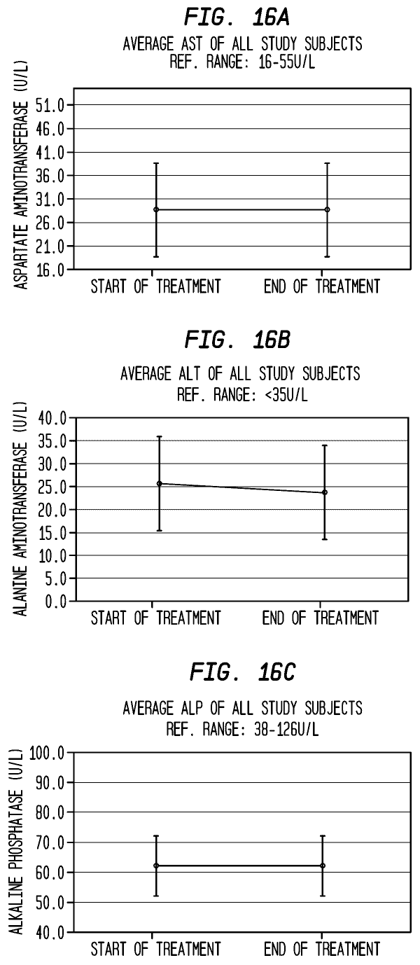

Now, with reference to FIGS. 16A, 16B, and 16C, liver function studies including alkaline phosphatase (ALP), alanine aminotransferase (ALT) and aspartate aminotransferase (ASTZ) across the 15 subjects in the study population over the two month treat period changed very minimally (p-value>0.05) and stayed within the normative range.

There were no adverse events. No patient experienced nausea, diarrhea, abdominal cramping, or pain. One patient reported transient tingling upon application. There were no instances of skin rash, erythema, itching, or hives. No patient reported an allergic reaction.

As can be easily understood from the foregoing, the basic concepts of the present invention may be embodied in a variety of ways. The invention involves numerous and varied embodiments of a topical composition and methods for making and using such topical composition including the best mode.

As such, the particular embodiments or elements of the invention disclosed by the description or shown in the figures or tables accompanying this application are not intended to be limiting, but rather illustrative of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled.

In addition, as to each term used it should be understood that unless its utilization in this application is inconsistent with such interpretation, common dictionary definitions should be understood to be included in the description for each term as contained in Merriam-Webster's Collegiate Dictionary, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the dry ingredient and carrier compositions herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A composition including an admixture of dry ingredients, comprising:
gynostemma powder extract;
sage extract;
cardamom powder extract;
coffee robusta extract; and
notoginseng extract.

2. The composition of claim 1, wherein said admixture of dry ingredients by weight percent, comprising:

| | |
|---|---|
| gynostemma powder extract | about 39.0% to about 43.0%; |
| sage extract | about 21.0% to about 25.0%; |
| cardamom powder extract | about 16% to about 20%; |
| coffee robusta extract | about 11.0% to about 15%; and |
| notoginseng extract | about 3.0% to about 7.0%. |

3. The composition of claim 1, further comprising a carrier composition, wherein said admixture of dry ingredients blended into said carrier composition, wherein weight percent of said admixture of dry ingredients in said carrier composition adjusted to effect, upon application to a skin area, a reduction in visceral fat deposition and/or preserve or enhance muscle mass in a body of a subject.

4. The composition of claim 3, wherein about 0.1 grams to about 0.5 grams of said admixture of dry ingredients included in each one milliliter of said carrier composition.

5. The composition of claim 1, wherein said admixture of dry ingredients, further comprising one or more of:
mulberry leaf extract;
goji berry powder extract; and
astragalus root extract.

6. The composition of claim 5, wherein said admixture of dry ingredients by weight percent, comprising:

| | |
|---|---|
| gynostemma powder extract | about 29.0% to about 34.0%; |
| mulberry leaf extract | about 16.0% to about 19.0%; |
| goji berry powder extract | about 12.0% to about 16.0%; |
| sage extract | about 10.0% to about 14%; |
| coffee robusta extract | about 8.0% to about 12%; |
| cardamom seed extract | about 6.0% to about 10%; |
| astragalus root extract | about 2.0% to about 6.0%; and |
| panax notoginseng root extract | about 2.0% to about 6.0%. |

7. The composition of claim 5, wherein said admixture of dry ingredients by weight percent consists essentially of:

| | |
|---|---|
| gynostemma powder extract | about 29.0% to about 34.0%; |
| mulberry leaf extract | about 16.0% to about 19.0%; |
| goji berry powder extract | about 12.0% to about 16.0%; |
| sage extract | about 10.0% to about 14%; |
| coffee robusta extract | about 8.0% to about 12%; |
| cardamom seed extract | about 6.0% to about 10%; |
| astragalus root extract | about 2.0% to about 6.0%; and |
| panax notoginseng root extract | about 2.0% to about 6.0%. |

8. The composition of claim 5, wherein said admixture of dry ingredients by weight percent consists of:

| | |
|---|---|
| gynostemma powder extract | about 29.0% to about 34.0%; |
| mulberry leaf extract | about 16.0% to about 19.0%; |
| goji berry powder extract | about 12.0% to about 16.0%; |
| sage extract | about 10.0% to about 14%; |
| coffee robusta extract | about 8.0% to about 12%; |
| cardamom seed extract | about 6.0% to about 10%; |
| astragalus root extract | about 2.0% to about 6.0%; and |
| panax notoginseng root extract: | about 2.0% to about 6.0%. |

9. The composition of claim 5, wherein said admixture of dry ingredients by weight percent comprises:

| | |
|---|---|
| gynostemma powder extract | about 31.4%; |
| mulberry leaf extract | about 17.7%; |
| goji berry powder extract | about 13.7%; |
| sage extract | about 11.8%; |
| coffee robusta extract | about 9.8%; |
| cardamom seed extract | about 7.8%; |
| astragalus root extract | about 3.9%; and |
| panax notoginseng root extract | about 3.9%. |

10. The composition of claim 5, wherein said percent by weight of said dry ingredients consists essentially of:

| | |
|---|---|
| gynostemma powder extract | about 31.4%; |
| mulberry leaf extract | about 17.7%; |
| goji berry powder extract | about 13.7%; |
| sage extract | about 11.8%; |
| coffee robusta extract | about 9.8%; |
| cardamom seed extract | about 7.8%; |
| astragalus root extract | about 3.9%; and |
| panax notoginseng root extract | about 3.9%. |

11. The composition of claim 5, wherein said percent by weight of said admixture of dry ingredients consists of:

| | |
|---|---|
| gynostemma powder extract | about 31.4%; |
| mulberry leaf extract | about 17.7%; |
| goji berry powder extract | about 13.7%; |
| sage extract | about 11.8%; |
| coffee robusta extract | about 9.8%; |
| cardamom seed extract | about 7.8%; |
| astragalus root extract | about 3.9%; and |
| panax notoginseng root extract | about 3.9%. |

12. The composition of claim 5, further comprising a carrier composition, wherein said admixture of dry ingredients blended into said carrier composition, wherein weight percent of said admixture of dry ingredients in said carrier composition adjusted to effect, upon application to a skin area, a reduction in visceral fat deposition and/or preserve or enhance muscle mass in a body of a subject.

13. The composition of claim 12, wherein weight percent of said admixture of dry ingredients relative to said carrier composition:

| | |
|---|---|
| gynostemma powder extract | about 0.5% to about 2.5%; |
| mulberry leaf extract | about 0.1% to about 1.0%; |
| goji berry powder extract | about 0.1% to about 0.8%; |
| sage extract | about 0.3% to about 1.5%; |
| coffee robusta extract | about 0.2% to about 1.1%; |
| cardamom seed extract | about 0.4% to about 1.5%; |
| astragalus root extract | about 0.1% to about 0.8%; and |
| panax notoginseng root extract | about 0.1% to about 0.8%. |

14. The composition of claim 12, wherein said admixture of dry ingredients blended in said carrier composition in milligrams per milliliter, comprising:

| | |
|---|---|
| gynostemma powder extract | about 35 milligrams to about |
| | 45 milligrams; |
| mulberry leaf extract | about 10 milligrams to about |
| | 20 milligrams; |
| goji berry powder extract | about 5 milligrams to about |
| | 15 milligrams; |
| sage extract | about 15 milligrams to about |
| | 25 milligrams; |
| coffee robusta extract | about 10 milligrams to about |
| | 20 milligrams; |
| cardamom seed extract | about 15 milligrams to about |
| | 25 milligrams; |
| astragalus root extract | about 2 milligrams to about |
| | 7 milligrams; and |
| panax notoginseng root extract | about 2 milligrams to about |
| | 7 milligrams. |

15. The composition of claim 12, wherein said admixture of dry ingredients blended in said carrier composition in milligrams per milliliter, comprising:

| | |
|---|---|
| gynostemma powder extract | about 40 milligrams; |
| mulberry leaf extract | about 15 milligrams; |
| goji berry powder extract | about 10 milligrams; |
| sage extract | about 23 milligrams; |
| coffee robusta extract | about 13 milligrams; |
| cardamom seed extract | about 18 milligrams; |
| astragalus root extract | about 5 milligrams; and |
| panax notoginseng root extract | about 5 milligrams. |

16. The composition of claim 12, wherein said admixture of dry ingredients blended in said carrier composition in milligrams per milliliter, consisting essentially of:

| | |
|---|---|
| gynostemma powder extract | about 40 milligrams; |
| mulberry leaf extract | about 15 milligrams; |
| goji berry powder extract | about 10 milligrams; |
| sage extract | about 23 milligrams; |
| coffee robusta extract | about 13 milligrams; |
| cardamom seed extract | about 18 milligrams; |
| astragalus root extract | about 5 milligrams; and |
| panax notoginseng root extract | about 5 milligrams. |

17. The composition of claim 12, wherein said admixture of dry ingredients blended in said carrier composition in milligrams per milliliter, consists of:

| | |
|---|---|
| gynostemma powder extract | about 40 milligrams; |
| mulberry leaf extract | about 15 milligrams; |
| goji berry powder extract | about 10 milligrams; |
| sage extract | about 23 milligrams; |
| coffee robusta extract | about 13 milligrams; |
| cardamom seed extract | about 18 milligrams; |
| astragalus root extract | about 5 milligrams; and |
| panax notoginseng root extract | about 5 milligrams. |

18. The composition of claim 3, wherein said carrier composition adapted to apply to a subject's skin.

19. The composition of claim 18, wherein said admixture of dry ingredients blended in said carrier composition comprises a topical composition effective upon application to a subject's skin to a reduce visceral fat deposition and/or preserve or enhance muscle mass in a body of a subject.

20. The composition of claim 19, wherein carrier composition, comprising:
carbomer;
glycerin;
polysorbate 20; and
water.

21. The composition of claim 20, wherein said carrier composition ingredients by weight percent, comprising:

| | |
|---|---|
| carbomer | about 2% to about 6%; |
| glycerin | about 5% to about 15%; |
| polysorbate 20 | about 1% to about 4%; and |
| water | the remaining weight percent to 100%. |

22. The composition of claim 20, wherein said carrier composition ingredients by weight percent including:

| | |
|---|---|
| carbomer | about 2% to about 6%; |
| glycerin | about 5% to about 15%; |
| polysorbate 20 | about 1% to about 4%; and |
| water | the remaining weight percent to 100%, |

wherein said carrier composition ingredients comprises a Phase A carrier composition.

23. The composition of claim 20, wherein said carrier composition ingredients further comprising one or more of:
emulsifying wax;
grape seed oil;
Acticire® MB;
Emulium® Dolcea;
polysorbate 80; and
Emulium® Mellifera.

24. The composition of claim 23, wherein said carrier composition ingredients by weight percent including:

| | |
|---|---|
| emulsifying wax | about 2% to about 5%; |
| grape seed oil | about 3% to about 8%; |
| Acticire ® MB | about 2% to about 7%; |
| Emulium ® Dolcea | about 1% to about 6%; |
| polysorbate 80 | about 1% to about 5%; and |
| Emulium ® Mellifera | about 0.5% to about 5%. |

25. The composition of claim 23, wherein said carrier composition ingredients by weight percent including:

| | |
|---|---|
| emulsifying wax | about 2% to about 5%; |
| grape seed oil | about 3% to about 8%; |
| Acticire ® MB | about 2% to about 7%; |
| Emulium ® Dolcea | about 1% to about 6%; |
| polysorbate 80 | about 1% to about 5%; |
| Emulium ® Mellifera | about 0.5% to about 5%, |

wherein said carrier composition ingredients comprises a Phase B carrier composition.

26. The composition of claim 25, wherein said carrier composition comprises said Phase A carrier composition combined with said Phase B carrier composition.

27. The composition of claim 26, wherein said admixture of dry ingredients blended into said Phase A carrier composition combined with said Phase B carrier composition to produce a topical composition effective to reduce visceral fat deposition upon application to skin of a subject and/or preserve or enhance muscle mass in a body of a subject.

28-32. (canceled)
